(19) United States
(12) Patent Application Publication
(10) Pub. No.: US 2009/0254116 A1
(43) Pub. Date: Oct. 8, 2009

(54) RETRIEVAL CATHETER AND METHODS OF RETRIEVING DEPLOYED MEDICAL DEVICES

(75) Inventors: Uri Rosenschein, M.P. Merkaz (IL); Benjamin Spenser, M.P. Hof Karmel (IL)

Correspondence Address: RISSMAN HENDRICKS & OLIVERIO, LLP, 100 Cambridge Street, Suite 2101, BOSTON, MA, 02114, US

(73) Assignee: Gardia Medical Ltd., Caesarea, IL (US)

(21) Appl. No.: 12/417,216

(22) Filed: Apr. 2, 2009

Related U.S. Application Data
(60) Provisional application No. 61/042,131, filed on Apr. 3, 2008.

(51) Int. Cl. A61M 25/09; A61B 17/22

(52) U.S. Cl. 606/200; 604/528

(57) ABSTRACT

A retrieval catheter for retrieving deployed medical devices includes a first guidewire lumen and a second guidewire lumen spaced radially from the first guidewire lumen. The first guidewire lumen has a distal end portion configured to recapture a medical device, such as a collapsed embolic protection device, deployed in a body lumen and secured to a guidewire extending through the first guidewire lumen. The second guidewire lumen receives a second guidewire which may be advanced past a first interventional site to perform a second procedure. The retrieval catheter may be provided with a soft tip to reduce trauma to the body tissue.

### CROSS-REFERENCE TO RELATED APPLICATIONS

This application claims priority to U.S. Provisional Application Ser. No. 61/042,131, filed Apr. 3, 2008, the entire content of which is incorporated herein by reference.

### TECHNICAL FIELD

The present invention is directed generally to catheters and retrieval methods. More particularly, the present invention is directed to dual-lumen retrieval catheters and methods of retrieving deployed medical devices such as, for example, embolic protection devices.

### BACKGROUND

Transcatheter procedures are employed in increasing numbers for opening stenosed or occluded blood vessels in patients caused by deposits of plaque or other materials on the walls of the blood vessels. Such minimally invasive procedures have proven to be advantageous compared to traditional surgical procedures, such as open heart surgery. Stenosis in arteries and other blood vessels can be treated by permanently or temporarily introducing a stent into the stenosed region to open the lumen of the vessel.

However, embolic material may be released into the blood stream during implantation of a stent or another prosthetic device, placing the patient at great risk. Embolic material formed of calcium deposits, intimal debris, pieces of artheromatous plaque and/or thrombi has the potential of migrating downstream and causing distal tissue damage, for example stroke or myocardial infarction (see Topol, E. J. and Yadav, J. S., “Recognition of the Importance of Embolization in Athereosclerotic Vascular Disease”, Circulation 2000, 101:570). Embolic material which can potentially damage the distal tissue is often released during vascular intervention procedures, such as stenting of an artheromatous region.

To alleviate this problem, an embolic filter, or other type of embolic protection device (EPD), may be advanced to a site distal to the treatment site to filter and capture undesired embolic material from the blood. The filter is typically inserted over or together with a guidewire using a delivery catheter. Following the treatment procedure, the filter is collapsed and removed from the body over the guidewire or together with the guidewire. Additional treatment devices, such as balloons and stents, can be inserted and/or removed via the same guidewire.

During some procedures, after treatment with a balloon and/or stent is completed, the surgeon may discover another lesion or stenosis distal of the original treatment site or discover a vessel dissection resulting from the immediately preceding procedure. In such cases, the surgeon must remove the guidewire with the collapsed filter and then re-deploy a new guidewire for treatment of the second site. This procedure of re-inserting a secondary guidewire adds significantly to the patient's treatment time since deployment of a guidewire is very time consuming. Additionally, re-inserting a secondary guidewire also introduces modest risk of plaque embolization while the guidewire is being position at the diseased artery.

Referring to FIG. 1, a deployed embolic protection device 10, for example, an embolic filter, is illustrated just distal of a first deployment site 13 of a stent 14. The device 10 is mounted on a guidewire 12 and is shown in an open (deployed) position. The device 10 may include an EPD with a guidewire locking mechanism of the type described in U.S. patent application Ser. No. 11/873,882, filed on Oct. 17, 2007, and entitled “Guidewire Stop,” and U.S. patent application Ser. No. 11/873,893, filed on Oct. 17, 2007, and entitled “Guidewire Stop,” the disclosures of which are incorporated herein by reference. Consequently, the removal of EPD 10 may require simultaneous retrieval of the guidewire 12 on which the EPD is lockingly deployed.

Thus, if a second lesion 17 is subsequently discovered, the surgeon would need to retrieve the EPD 10 together with the guidewire 12, and subsequently replace the guidewire 12 with a new one. For such cases, it would be desired to provide a retrieval catheter that permits delivery of a secondary guidewire while the primary guidewire 12 and EPD 10 are still deployed. In particular, it would be desirable if the retrieval catheter could also serve to introduce a secondary guidewire, prior to recovery of the primary guidewire and EPD. Thus, such an improved retrieval catheter would perform the dual functions of delivery of a secondary guidewire while retrieving a previously-deployed EPD system.

Retrieving an EPD has posed challenges because of apparently contradictory requirements for the design of the retrieval catheter. On one hand, the collecting tube with which the filter is retrieved needs to have a large inside diameter in order to capture the filter easily and avoid squeezing out the material caught in the filter during the procedure; on the other hand, the size of the collecting tube is limited by the size of the guiding catheter size, the stent size through which the collecting tube must navigate, trauma to vessel, etc.

A large open-tube design of the collecting tube can be traumatic to the blood vessel. To alleviate trauma and facilitate navigation, the distal part of, for example, balloon catheters has a tapered soft tip which tapers all the way from the maximal tube diameter to the guidewire diameter. In this way, the catheter does not “dig” into the vessel wall, especially in tortuous vessels or get caught when traversing a stent, where struts may protrude toward the center of the blood vessel.

Several manufacturers have attempted to solve this problem by tapering the distal end of the collecting tube used for retrieval which, however, tends to make retrieval of the full filter more difficult and risks discharging particles back into the blood stream. In addition, the collecting tube and catheter need to be built from a sufficiently rigid material to prevent collapse when the filter is pulled into the tube, which makes it impossible to use a very flexible tube which will track softly through the blood vessels.

The retrieval catheters and retrieval methods of the present disclosure solve one or more of the problems set forth above.

### SUMMARY OF THE INVENTION

According to one aspect of the invention, a catheter for retrieving a medical device deployed in a body lumen includes a first guidewire lumen extending through a distal shaft portion of the retrieval catheter from a first proximal guidewire port to a first distal guidewire port, and a second guidewire lumen extending through the distal shaft portion of the retrieval catheter from a second proximal guidewire port to a second distal guidewire port. The first guidewire lumen has a distal end portion dimensioned to recapture the medical device secured on a first guidewire distal of the distal end portion. The first guidewire passes through the first guidewire lumen. The second guidewire lumen is radially offset from the first guidewire lumen and receives a second guidewire operable separately from the first guidewire for advancement past the distal end portion following recapture of the medical device.

According to another aspect of the invention, a method of retrieving an embolic protection device deployed in a body lumen at a location distal to a first interventional procedure site and secured to a first guidewire includes the steps of advancing a retrieval catheter along the first guidewire to a location proximal of the embolic protection device, wherein the retrieval catheter has a first guidewire lumen extending through a distal shaft portion of the retrieval catheter from a first proximal guidewire port to a first distal guidewire port. The first guidewire lumen has a distal end portion dimensioned to recapture the embolic protection in a collapsed state, and the first guidewire passes through the first guidewire lumen. The retrieval catheter further has a second guidewire lumen extending through the distal shaft portion of the retrieval catheter from a second proximal guidewire port to a second distal guidewire port. The second guidewire lumen is radially offset from the first guidewire lumen and receives a second guidewire operable separately from the first guidewire. The method includes the additional steps of recapturing the collapsed embolic protection device into the first guidewire lumen of the retrieval catheter, advancing the second guidewire through the second guidewire lumen of the retrieval catheter to a second interventional procedure site, and withdrawing the retrieval catheter with the embolic protection device and the first guidewire, while leaving the second guidewire in place.

According to one embodiment, the dual-lumen retrieval catheter may include a tipped member having a conical shape with a greatest outside diameter sized to be slidably received in the distal end portion of the first guidewire lumen and a center opening through which the first guidewire passes. The tip of the tipped member may protrude distally from the distal end portion when the retrieval catheter is advanced distally in the body lumen towards the medical device to prevent trauma to the vessel.

According to yet another aspect of the invention, a catheter for retrieving a medical device deployed in a body lumen includes a guidewire lumen extending through a distal shaft portion of the retrieval catheter from a first proximal guidewire port to a first distal guidewire port. The guidewire lumen has a distal end portion dimensioned to recapture the medical device secured on a guidewire distal of the distal end portion. The guidewire passes through the guidewire lumen. The catheter further includes a tipped member having a conical shape with a greatest outside diameter sized to be slidably received in the distal end portion of the guidewire lumen and a center opening through which the guidewire passes. The tip of the tipped member protrudes distally from the distal end portion when the retrieval catheter is advanced distally in the body lumen towards the medical device.

According to still another aspect of the invention, a method of retrieving an embolic protection device deployed in a body lumen and secured to a guidewire includes the steps of advancing a retrieval catheter along the guidewire to a location proximal of the embolic protection device. The retrieval catheter has a guidewire lumen extending through a distal shaft portion of the retrieval catheter from a proximal guidewire port to a distal guidewire port. The guidewire lumen has a distal end portion dimensioned to recapture the embolic protection device in a collapsed state. A tipped member having a conical shape is slidably received in a distal end portion of the guidewire lumen, with the pointed end protruding distally from the distal end portion. The guidewire passes longitudinally through the tipped member. The method includes the additional steps of collapsing the embolic protection device, and pulling the collapsed embolic protection device proximally into the guidewire lumen of the retrieval catheter.

In one embodiment, a pulling wire may be secured to the tipped member, for example by a material connection or a force-transmitting connection, for defining a longitudinal position of the tipped member inside the receiving guidewire lumen. The tipped member may frictionally engage with the distal end portion of the receiving guidewire lumen.

In one embodiment, a proximal actuator may be provided which has a distal section attached to a proximal shaft portion of the retrieval catheter, and a proximal section movable longitudinally with respect to the distal section and secured to the pulling wire. The tipped member is longitudinally displaced inside the distal end portion of the first guidewire lumen by moving the proximal section relative to the distal section.

The retrieval catheter may be configured for rapid exchange.

These and other features and advantages of the present invention will become more readily appreciated from the detailed description of the invention that follows.

### BRIEF DESCRIPTION OF THE DRAWINGS

The following figures depict certain illustrative embodiments of the invention in which like reference numerals refer to like elements. These depicted embodiments are to be understood as illustrative of the invention and not as limiting in any way.

FIG. 1 is a schematic view of an exemplary embolic protection device deployed in a body lumen;

FIG. 2 is a schematic view of an exemplary dual-lumen retrieval catheter deployed in a body lumen in accordance with various aspects of the invention;

FIG. 3 is a schematic view of a guidewire deployed in a body lumen

FIG. 4A is a schematic view of an exemplary dual-lumen retrieval catheter deployed in a body lumen in accordance with various aspects of the invention,

FIGS. 4B and 4C are schematic views of a proximal end of an exemplary retrieval catheter in accordance with various aspects of the invention,

FIG. 5 is a schematic view of an exemplary dual-lumen retrieval catheter in accordance with various aspects of the invention,

FIG. 6 shows schematically a conventional retrieval catheter passing through a stented region of a vessel,

FIG. 7 is a schematic view of an exemplary retrieval catheter having a soft distal tip in accordance with various aspects of the invention,

FIGS. 8A-C show schematically different stages of retrieval of an embolic filter,

FIGS. 9A and B show schematically in more detail the mechanism employed with the retrieval catheter for retrieving an embolic filter,

FIG. 10A is a schematic view of the dual-lumen retrieval catheter of FIG. 5A with a soft tip to ensure non-traumatic passage through a vessel lumen, and

FIG. 10B shows schematically an embolic protection device following retrieval from the vessel with the dual-lumen retrieval catheter of FIG. 5B.

### DETAILED DESCRIPTION

Various aspects of exemplary retrieval catheters and methods are disclosed herein which may efficiently and effectively enable a therapeutic procedure to be performed in a blood vessel at an interventional procedure site, for example, stenosis site due to plaque. The exemplary catheters and methods may be part of a therapeutic system and method configured to occlude the blood vessel at a location relative to the interventional procedure site, prevent the flow of blood past the occlusion, and enable the capture and recovery of embolic material which may be released into the blood vessel during the interventional procedure.

The retrieval catheters and methods are illustrated and described herein by way of example only and not by way of limitation. While the exemplary retrieval catheters and methods are described in detail as applied to the carotid arteries of the patient, those skilled in the art will appreciate that they can also be used in other body lumens as well, such as the coronary arteries, renal arteries, saphenous veins, and other peripheral arteries. Additionally, the exemplary retrieval catheters and methods can be utilized when performing any one of a number of interventional procedures, such as stenting, balloon angioplasty, laser angioplasty, or atherectomy.

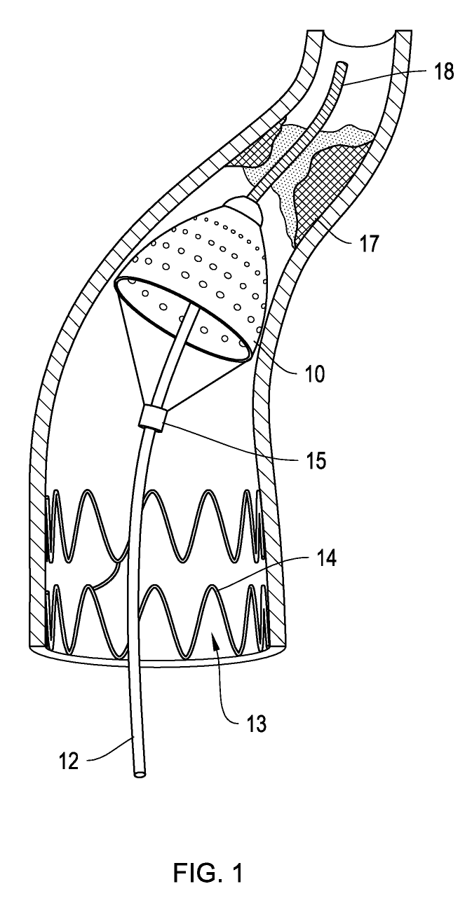

Referring again back to FIG. 1, a primary guidewire 12, for example a guidewire used with a rapid exchange delivery and retrieval system, may be delivered to a first interventional procedure site 13. As would be appreciated by persons skilled in the art, in accordance with conventional intravascular procedures using the well-known Seldinger Method, a Seldinger wire and introducer sheath may be inserted into the femoral artery at a patient's groin area. A guide catheter (not shown) can then be inserted into the femoral artery via the introducer sheath, with the distal end of the guide catheter ultimately positioned at a location proximal of the interventional procedure site 13. For example, in procedures involving the carotid arteries, the guide catheter may be directed through the descending aorta to the aortic arch, with the distal end of the guide catheter being positioned such that the distal end thereof is fixedly positioned and intubating an ostium associated with the desired common carotid artery requiring access and treatment.

The primary guidewire 12 may then be controllably steered to the site 13 of a lesion or stenosis in a body lumen, for example, a blood vessel, following introduction through a pre-deployed guide catheter. Primary guidewire 12 is manipulated the interventional vascular practitioner into the selected left or right common carotid artery, as appropriate. Primary guidewire 12 is then steered into an appropriately selected internal or external carotid artery, in accordance with the location of the procedure site 13. Ultimately, the distal tip of primary guidewire 12 is positioned at a location slightly distal of the interventional procedure site 13. Primary guidewire 12 can then be used to deliver an EPD 10, such as an embolic protection filter, to a location slightly distal of the interventional procedure site 13. EPD 10 may preferably be delivered to the treatment site using a rapid exchange embolic protection device delivery catheter (not shown). As previously discussed, the embolic protection device 10 may be configured to lock onto the guidewire 12, for example, as described in U.S. patent application Ser. Nos. 11/873,882 and 11/873,893.

The intravascular therapeutic treatment procedure may further include insertion of a balloon dilatation catheter followed by a stent delivery catheter, both catheters utilizing a rapid exchange configuration for ease of access to the site of the stenosis or lesion to be treated. Since all of the aforementioned catheters preferably utilize the rapid exchange configuration, each catheter can easily be substituted and introduced over a standard length interventional guidewire. The balloon and stent catheters are delivered over the guidewire to a position just proximal of the deployed embolic protection device. The balloon and stent may be deployed to treat the stenosis or lesion, as is known by persons skilled in the art. As previously mentioned, if the surgeon notices a second treatment site distal to the first site after the first site has been treated, a dual-lumen retrieval device in accordance with the disclosure may then be delivered to capture the embolic protection device, assist in delivery of a secondary guidewire, and remove the primary guidewire originally introduced, together with the re-captured embolic protection device, from the body lumen.

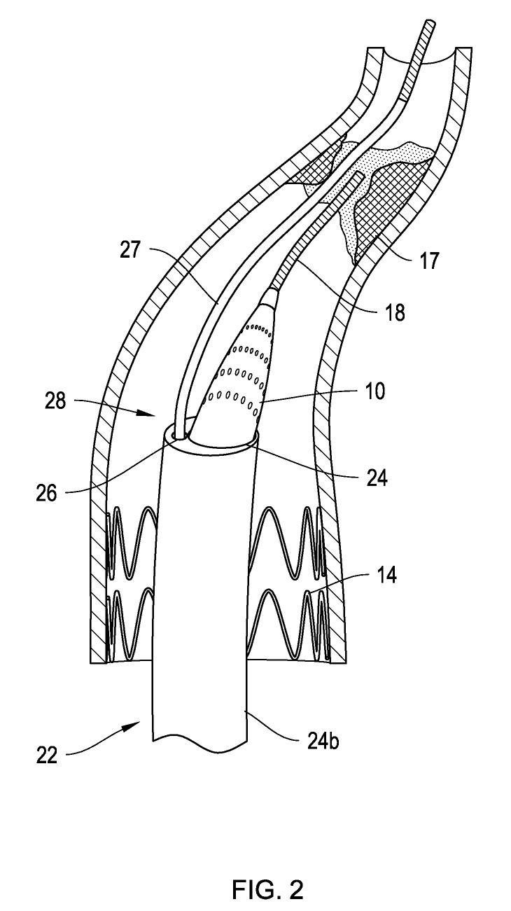

A distal portion of an exemplary embodiment of a dual-lumen retrieval catheter 22 is shown in FIG. 2. The retrieval catheter 22 includes a first lumen 24 and a second lumen 26. The first lumen 24 may be configured to receive the embolic protection device 10, e.g. a filter. For example, referring back to FIG. 1, the proximal collar 15 of the embolic protection device 10 may be fixedly coupled to the primary guidewire 12 to stabilize the embolic protection device 10 relative to the guidewire 12 such that the device 10 will collapse for recapture into the first lumen 24 at a distal end 28 of the retrieval catheter 22.

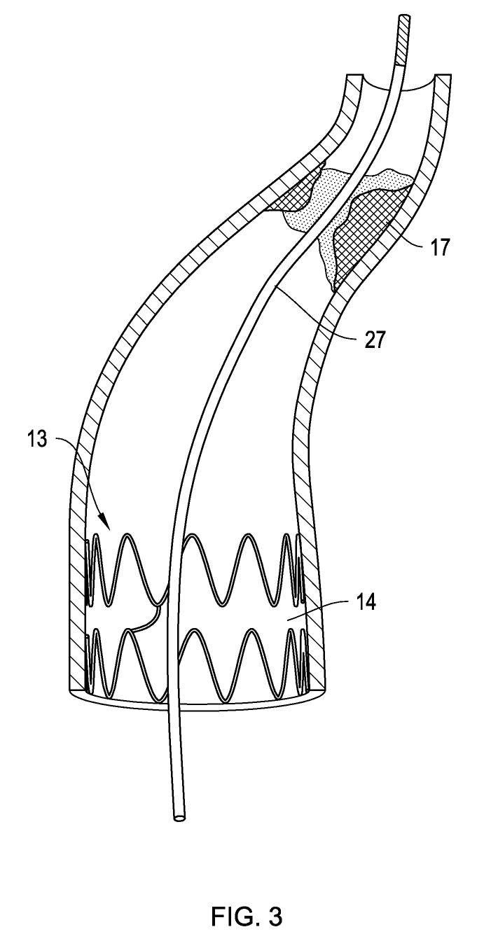

In operation, the retrieval catheter 22 may be delivered to the intravascular site where the embolic protection device 10 is deployed. The first lumen 24 of the catheter 22 receives a collapsing or collapsed EPD 10. As previously described, the embolic protection device 10 may be retrieved with the primary guidewire 18, upon which it is locked. Removal of the catheter 22 with EPD 10 leaves the secondary guidewire 27 in a therapeutic position associated with the second lesion site 17, as shown in FIG. 3

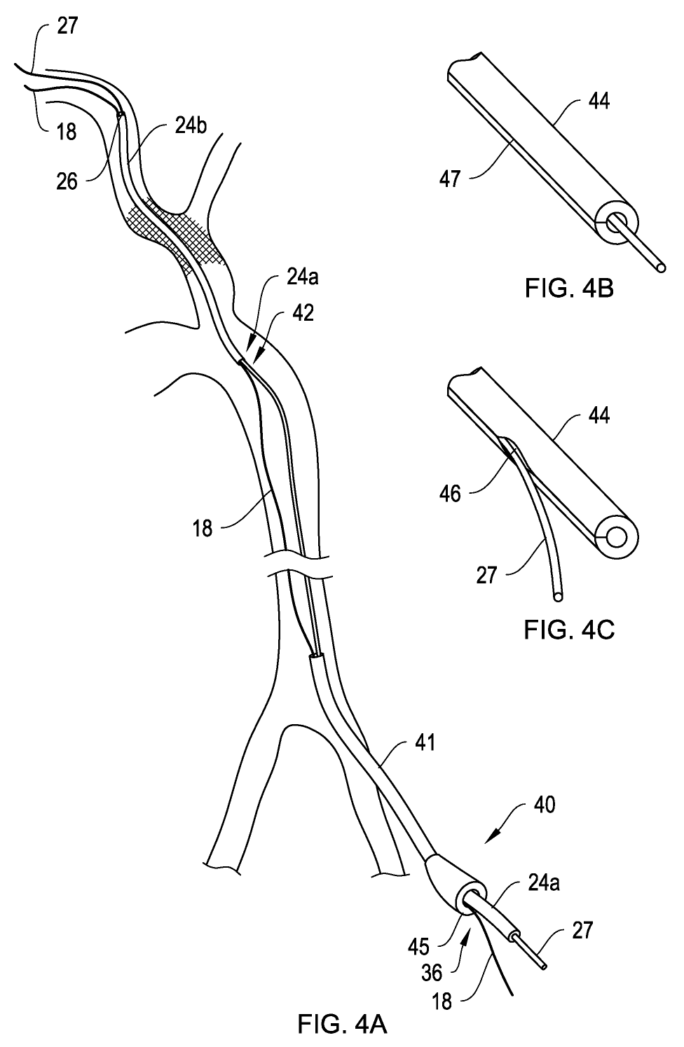

Referring now to FIG. 4A, the retrieval catheter 22 may include a rapid exchange (RX) retrieval catheter. The rapid exchange catheter 22 may include a proximal shaft portion 24a located at a proximal end 40 of the catheter 22 and a distal shaft portion 24b. The proximal shaft portion 24a may extend through a guide catheter 41 and preferably have a length sufficient to extend from the site of introduction at the femoral artery to an appropriate distal location such that a distal end of the guide catheter 41 for intubation into an appropriate ostium of the aortic arch. It will be appreciated that the guide catheter 41 may extend further than illustrated in FIG. 4A so as to enclose the primary guidewire 18 and the proximal shaft portion 24a within the guide catheter 41; however, for sake of clarity, these elements are illustrated as not being enclosed by the guide catheter 41. A hub 45 located at the proximal end of the guide catheter 41 facilitates longitudinal repositioning of the primary guidewire 18 and delivery catheters while maintaining hemostasis via a valve (not shown) on the hub 45.

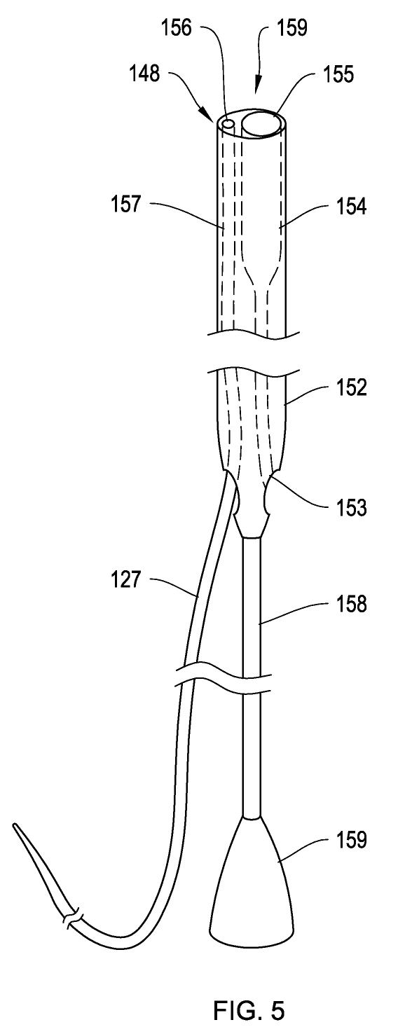

To enable rapid exchange, the distal shaft portion 24b includes a first proximal guidewire port 42, a first distal guidewire port 24, and a second distal guidewire port 26. The first proximal guidewire port 42 may be spaced from the proximal end 40 of the catheter 22. The first guidewire lumen 24 may extend through the distal shaft portion 24b from the first proximal guidewire port 42 to the first distal guidewire port 24. The first guidewire lumen 24 may have a distal end portion (not shown), which is similar to the collecting tube 55 shown in FIG. 5 and configured to recapture a collapsed embolic protection device 10. A second guidewire lumen may extend through the distal shaft portion 24b from a second proximal guidewire port 36 to the second distal guidewire port 26.

The primary guidewire 18, which extends distally from the distal shaft portion 24b, exits a proximal end 24a of the distal shaft portion at a rapid exchange port 42 and extends proximally parallel with the proximal shaft portion 24a and through the guide catheter 41 and a hub 45 as it exits the patient's body.

A secondary guidewire 27 can be introduced through the catheter 22 via the proximal shaft portion 24a and extends from the second lumen 26 of the distal shaft portion 24b to enter the patient's blood vessel distal of the catheter 22 (FIG. 2). The proximal shaft portion 24a may extend through the introducing sheath 41 and the hub 45. The secondary guidewire 27 may exit the patient's body via an exit port 48 of the proximal shaft portion.

According to some aspects, as shown in FIGS. 4B and 4C, for example, in order to retrieve the catheter 22 from the patient's body with a guidewire used in rapid exchange procedures, a shaft 44 of the proximal shaft portion 24a may include a slit 47 extending along its length, from the exit port 48 to the rapid exchange port 42. The slit 47 is configured to open and allow the catheter shaft 44 to be peeled 46 away from the secondary guidewire 27 during retrieval. Alternatively, a longer guidewire, such as a Kaltenbach wire, can be employed in lieu of a catheter with a slit when using a full-length over-the-wire retrieval catheter.

Another retrieval catheter and retrieval method is shown in and described with reference to FIG. 5. According to one aspect, a retrieval catheter 152 may include a first lumen 155 and a second lumen 156. As shown, a new guidewire 127 may be pre-loaded into catheter 152 prior to insertion of the catheter 152 into a patient's body. A distal tip 157 of the new guidewire 127 may be close to an exit port 148 of the second lumen 156.

The first lumen 155 may be configured to collect a deployed EPD 10 (not shown in FIG. 5) locked onto original guidewire 18 in collecting tube 154. The catheter 152 may include a rapid exchange port 153 extending from a proximal end of a distal shaft portion to a distal end 159 of the catheter. The catheter 152 may also include a handle 159 and a shaft 158 connecting the handle 159 to the catheter near the rapid exchange port 153.

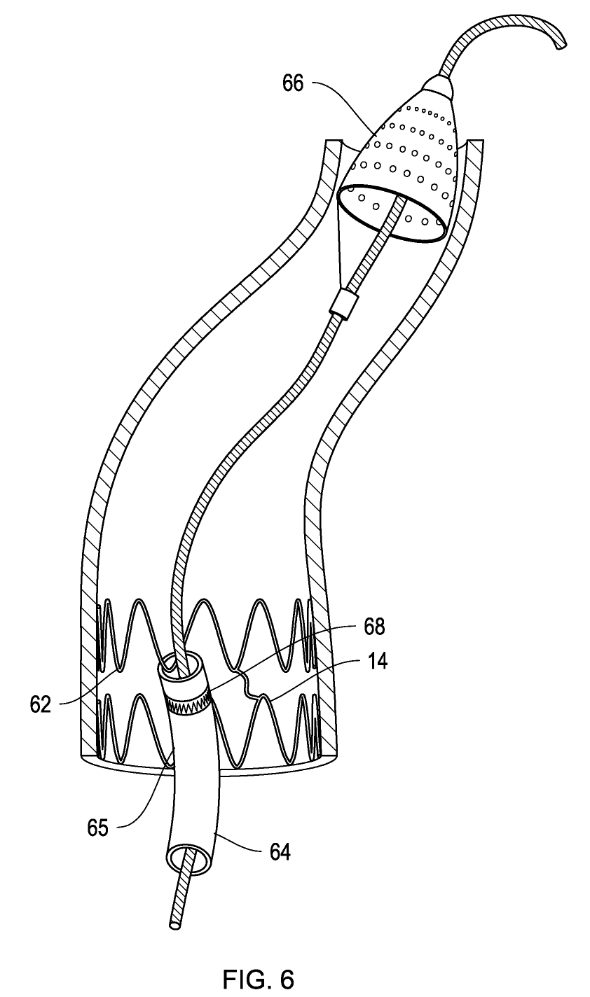

As evident from FIG. 5, the retrieval catheter 152 may not be able to traverse a stent 14 (FIG. 2) smoothly, and may easily get caught on protrusions 62 of stent 14, as depicted in FIG. 6. This is shown more clearly for an exemplary single tube retrieval or collection catheter 64 with an indicated radiopaque marker 68. When pushed from the proximal end, the tube may got caught on a protruding strut 62 of a stent 14 which interrupts passage of the retrieval catheter toward the EPD 66 to be retrieved. When the collection catheter 64 is pushed, it may bend at 65 when getting caught on the strut. Further attempts to push then may cause trauma to the blood vessel, especially at the stent position.

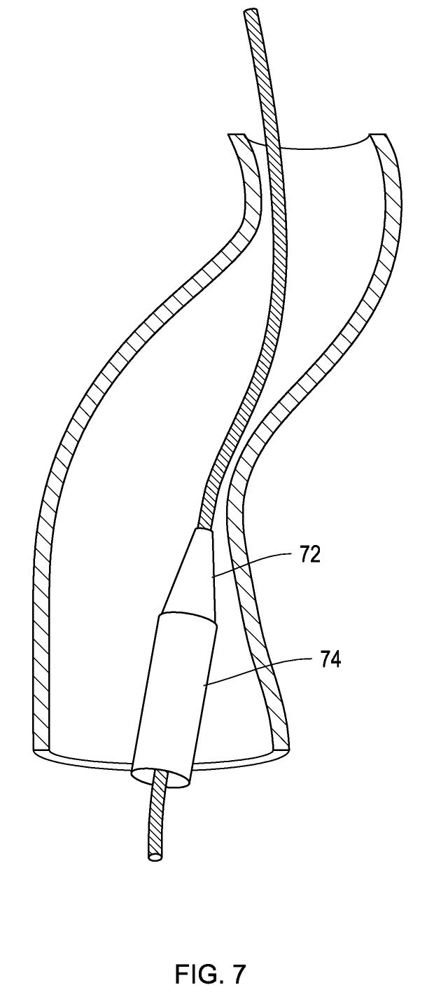

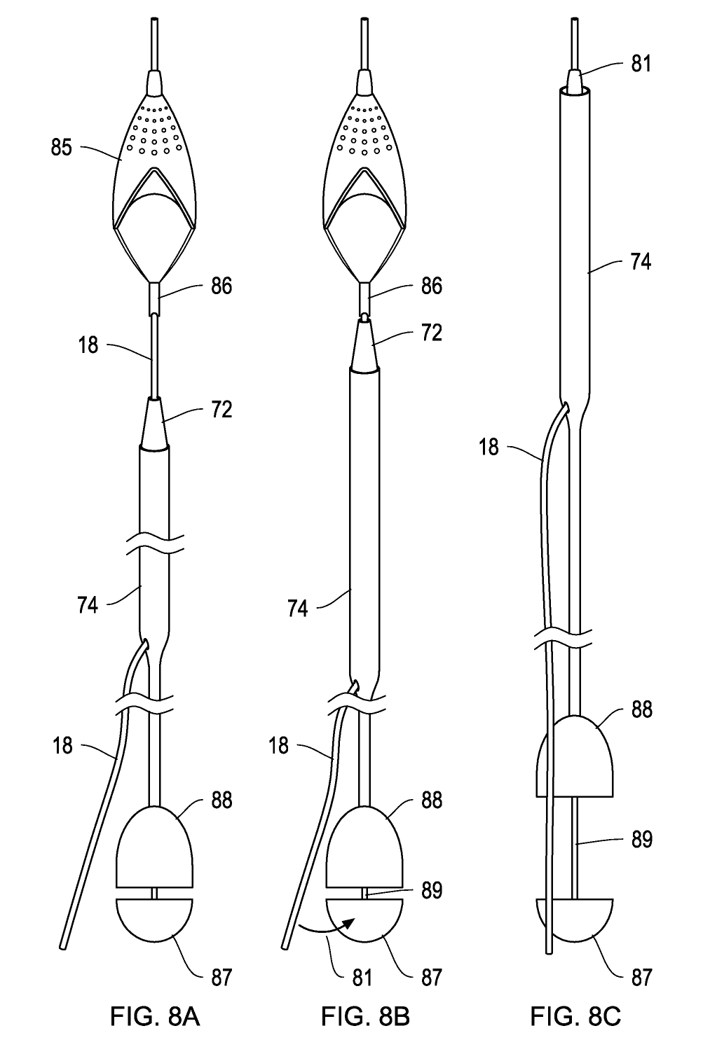

FIG. 7 shows a retrieval catheter 74 with a tapered soft tip 72 which will alleviate this problem and ensure a smooth and non-traumatic passage along the vessel wall. As indicated in FIG. 7 and described in more detail hereinafter with reference to FIGS. 8A-C, the greatest outside diameter of the tapered tip 72 is smaller than the inside diameter if the retrieval catheter, so that the tapered tip 72 is retracted into the retrieval catheter together with the EPD device to be retracted.

FIGS. 8A-C show different stages in the retrieval of an embolic protection device (EPD) 85 with the soft-tipped retrieval catheter 74 illustrated in FIG. 7. As seen in FIG. 8A, the retrieval catheter 74 is advanced distally over the guidewire 18 towards the EPD 85 until it comes in contact with the proximal side 86 of EPD 85. At this stage of the process, the guidewire 18 is coupled to or held, as illustrated in FIG. 8B by arrow 81, against the retrieval catheter's proximal handle section 87, which is slidably guided in or with respect to the retrieval catheter's distal handle section 88. The guidewire 18 may be coupled to proximal handle section 87 by frictional engagement, for example, with clips affixed to the outside of handle section 87, or the proximal handle section 87 may be constructed as a collet or chuck through which guidewire 18 passes.

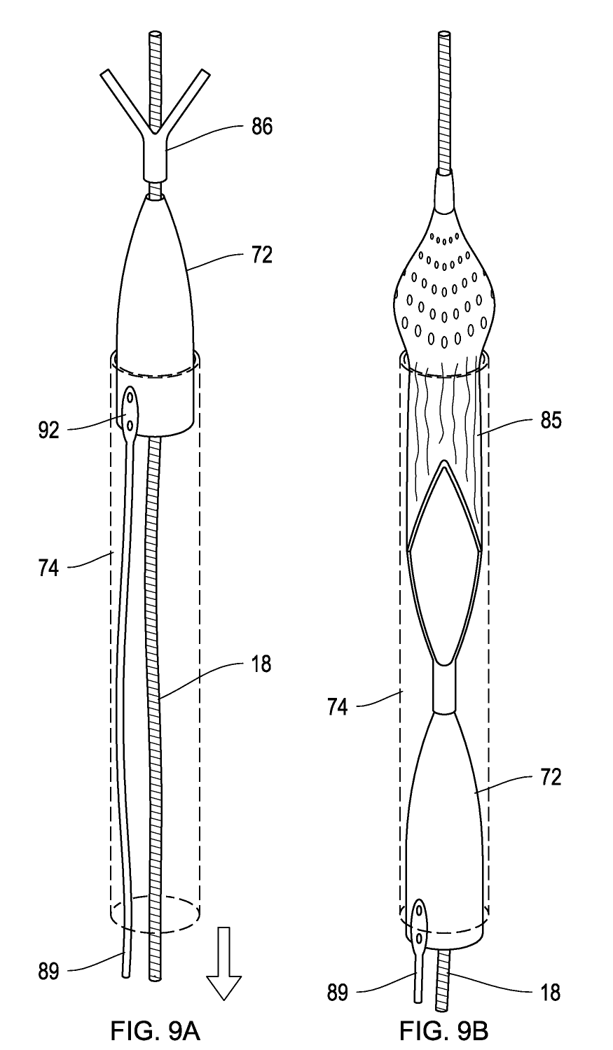

The guidewire 18 together with the proximal handle section 87 is then pulled proximally away from the stationary distal handle section 88, as shown in FIG. 8C, pulling the EPD 85 and the soft tip 72 into the retrieval catheter or collecting tube 74. FIG. 8C shows the filter (obscured from view) after retrieval inside the retrieval catheter or collecting tube 74, with only the conventional soft tip of the EPD 85 protruding from the distal end of the retrieval catheter or collecting tube 74. A pulling wire 89 is attached with its proximal end to the proximal handle section 87 and with its distal end to the proximal end of soft tip 72, as described in more detail with reference to FIG. 9. Pulling wire 89 may be a flexible wire and comprise a metal wire or polymer suture, for example.

FIGS. 9A and 9B show in more detail the mechanism by which the soft tip 72 is movably held inside the retrieval catheter 74. As shown in FIG. 9A, the soft tip 72 is attached at attachment point 92 to pulling wire 89 that extends through the retrieval catheter 74 to the proximal handle 87 (see also FIG. 8C). The pulling wire 89 can be attached to the soft tip 72 using various fastening methods, such as a material connection (brazing, welding), or a force-transmitting connection such as an adhesive or a rivet. After soft tip 72 comes in contact with the EDP 85, both the EPD 85 and the soft tip 72 are pulled out proximally, with the EPD 85 collapsing and assuming the position inside the retrieval catheter 74 indicated in FIG. 9B.

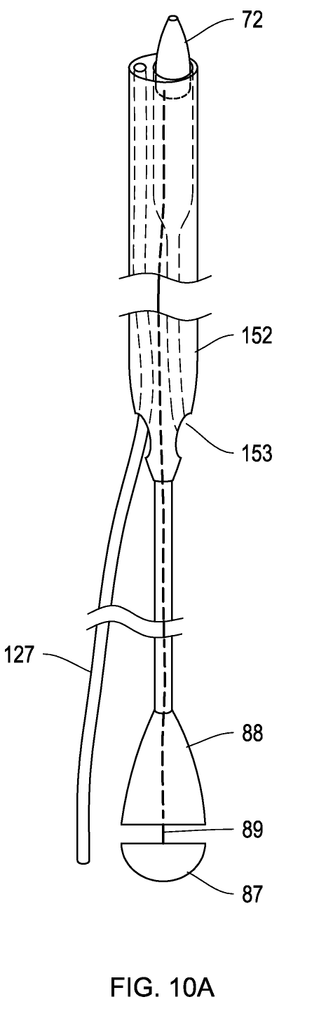

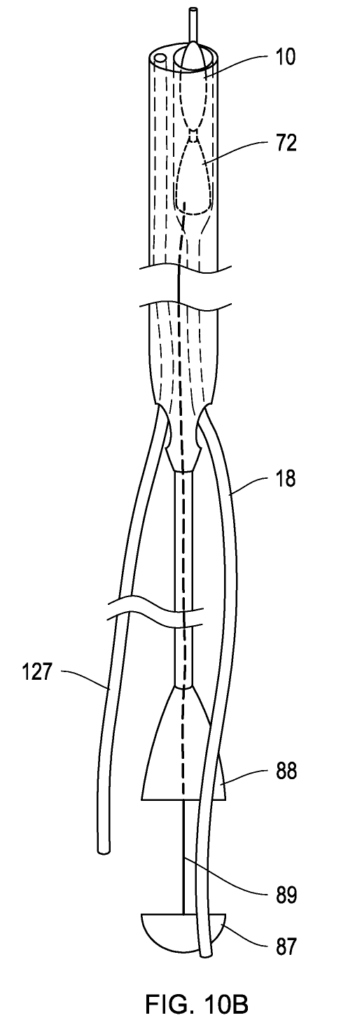

It will be appreciated by those skilled in the art that the soft-tipped retrieval catheter depicted in FIG. 7 can also be employed with the dual-lumen retrieval catheter illustrated in FIG. 5. Identical elements or elements performing substantially identical functions are indicated with the same reference numerals. The primary guidewire 18 has been omitted from FIG. 10A for sake of clarity, but is shown in FIG. 10B. With the soft tip 72 in place as shown, the dual-lumen catheter can be advanced through a vessel without causing trauma to the vessel walls. The retraction procedure for the EPD 10 is identical to the procedure described in FIGS. 8A-C for a single-lumen retrieval catheter.

Those of skill in the art will appreciate that the soft tip 72 can also be frictionally held in place inside the collection tube 74, obviating the need for a separate pull wire. Retraction of the EPD 85 will then also push the soft tip 72 back into the lumen of the retrieval catheter. However, this process may distort the shape of the soft tip 72, requiring a harder material for the soft tip 72 which could then again disadvantageously induce trauma in the vessel. In addition, the soft tip 72 would not be securely held captive in the collection tube 74 during advancement through the vessel and may become dislodged from its position inside the collection tube 74, though still being held by the guidewire extending through the soft tip 72.

It will be apparent to those skilled in the art that various modifications and variations can be made to the retrieval catheters and methods of the present invention without departing from the scope of the invention. Other embodiments of the invention will be apparent to those skilled in the art from consideration of the specification and practice of the invention disclosed herein. It is intended that the specification and examples be considered as exemplary only.

What is claimed is:

1. A retrieval catheter for retrieving a medical device deployed in a body lumen, comprising:
a first guidewire lumen extending through a distal shaft portion of the retrieval catheter from a first proximal guidewire port to a first distal guidewire port, the first guidewire lumen having a distal end portion dimensioned to recapture the medical device secured on a first guidewire distal of the distal end portion, said first guidewire extending through the first guidewire lumen; and
a second guidewire lumen extending through the distal shaft portion of the retrieval catheter from a second proximal guidewire port to a second distal guidewire port, said second guidewire lumen radially offset from the first guidewire lumen and receiving a second guidewire operable separately from the first guidewire for advancing past the distal end portion following recapture of the medical device.

2. The retrieval catheter of claim 1, further comprising:
a tipped member having a conical shape with a greatest outside diameter sized to be slidably received in the distal end portion of the first guidewire lumen and a center opening through which the first guidewire passes, wherein a tip of the tipped member protrudes distally from the distal end portion when the retrieval catheter is advanced distally in the body lumen towards the medical device.

3. The retrieval catheter of claim 2, further comprising:
a pulling wire secured to the tipped member for defining a longitudinal position of the tipped member inside the first guidewire lumen.

4. The retrieval catheter of claim 1, wherein the medical device comprises an embolic protection device.

5. The retrieval catheter of claim 3, further comprising:
a proximal actuator having a distal section attached to a proximal shaft portion of the retrieval catheter, and a proximal section movable longitudinally with respect to the distal section and secured to the pulling wire, wherein the tipped member is longitudinally displaced inside the distal end portion of the first guidewire lumen by moving the proximal section relative to the distal section.

6. The retrieval catheter of claim 1, wherein the retrieval catheter is configured for rapid exchange.

7. The retrieval catheter of claim 2, wherein the tipped member frictionally engages with the distal end portion of the first guidewire lumen.

8. A method of retrieving an embolic protection device deployed in a body lumen at a location distal to a first interventional procedure site and secured to a first guidewire, the method comprising the steps of:
advancing a retrieval catheter along the first guidewire to a location proximal of the embolic protection device, the retrieval catheter having a first guidewire lumen extending through a distal shaft portion of the retrieval catheter from a first proximal guidewire port to a first distal guidewire port, the first guidewire lumen having a distal end portion dimensioned to recapture the embolic protection in a collapsed state, said first guidewire extending through the first guidewire lumen, the retrieval catheter further having a second guidewire lumen extending through the distal shaft portion of the retrieval catheter from a second proximal guidewire port to a second distal guidewire port, said second guidewire lumen radially offset from the first guidewire lumen and receiving a second guidewire operable separately from the first guidewire;
recapturing the collapsed embolic protection device into the first guidewire lumen of the retrieval catheter;
advancing the second guidewire through the second guidewire lumen of the retrieval catheter to a second interventional procedure site; and
withdrawing the retrieval catheter with the embolic protection device and the first guidewire, while leaving the second guidewire in place.

9. The method of claim 8, further comprising the step of:
providing in the distal end portion of the first guidewire lumen a tipped member having a conical shape, with a pointed end of the tipped member protruding distally from the distal end portion.

10. The method of claim 9, wherein the tipped member frictionally engages with the distal end portion of the first guidewire lumen.

11. The method of claim 9, further comprising the step of:
withdrawing the tipped member into the distal end portion of the first guidewire lumen by proximally pulling with a pulling wire attached to the tipped member, while simultaneously recapturing the collapsed embolic protection device into the first guidewire lumen of the retrieval catheter.

12. A retrieval catheter for retrieving a medical device deployed in a body lumen, comprising:
a guidewire lumen extending through a distal shaft portion of the retrieval catheter from a first proximal guidewire port to a first distal guidewire port, the guidewire lumen having a distal end portion dimensioned to recapture the medical device secured on a guidewire distal of the distal end portion, said guidewire extending through the guidewire lumen; and
a tipped member having a conical shape with a greatest outside diameter sized to be slidably received in the distal end portion of the guidewire lumen and a center opening through which the guidewire passes, wherein a tip of the tipped member protrudes distally from the distal end portion when the retrieval catheter is advanced distally in the body lumen towards the medical device.

13. The retrieval catheter of claim 12, further comprising:
a pulling wire secured to the tipped member for defining a longitudinal position of the tipped member inside the guidewire lumen.

14. The retrieval catheter of claim 12, wherein the medical device comprises an embolic protection device.

15. The retrieval catheter of claim 12, wherein the tipped member frictionally engages with the distal end portion of the guidewire lumen.

16. The retrieval catheter of claim 12, wherein the pulling wire is secured to the tipped member by a material connection or a force-transmitting connection.

17. The retrieval catheter of claim 12, wherein the retrieval catheter is configured for rapid exchange.

18. A method of retrieving an embolic protection device deployed in a body lumen and secured to a guidewire, the method comprising the steps of:
advancing a retrieval catheter along the guidewire to a location proximal of the embolic protection device, the retrieval catheter having a guidewire lumen extending through a distal shaft portion of the retrieval catheter from a proximal guidewire port to a distal guidewire port, the guidewire lumen having a distal end portion dimensioned to recapture the embolic protection device in a collapsed state, and a tipped member having a conical shape slidably received in a distal end portion of the guidewire lumen and a pointed end protruding distally from the distal end portion, with the guidewire passing longitudinally through the tipped member,
collapsing the embolic protection device, and
pulling the collapsed embolic protection device proximally into the guidewire lumen of the retrieval catheter.

19. The method of claim 18, further comprising the steps of:
proximally pulling the tipped member into the guidewire lumen of the retrieval catheter with a pulling wire attached to the tipped member, and
simultaneously proximally pulling the collapsed embolic protection device into the guidewire lumen of the retrieval catheter with the guidewire so as to substantially retain the shape of the tipped member.
